(19) United States
(12) Patent Application Publication
(10) Pub. No.: US 2021/0177508 A1
(43) Pub. Date: Jun. 17, 2021

(54) DEVICES AND METHODS FOR THE CREATION OF AN INTER-ATRIAL SHUNT FOR THE TREATMENT OF CONGESTIVE HEART FAILURE

(71) Applicant: Avenu Medical, Inc., San Juan Capistrano, CA (US)

(72) Inventor: Brad M. Kellerman, Escondido, CA (US)

(21) Appl. No.: 17/119,027

(22) Filed: Dec. 11, 2020

Related U.S. Application Data
(60) Provisional application No. 62/947,416, filed on Dec. 12, 2019.

(51) Int. Cl. A61B 18/14

(52) U.S. Cl. CPC A61B 18/1492; A61B 2018/00577

(57) ABSTRACT

A system and method are adapted for use to reduce the left atrium pressure by creating an inter-atrial shunt (IAS) between the left and right atrium, thereby reducing pulmonary congestion and resulting in a reduction in pulmonary congestion, thereby reducing symptomatic dyspnea and increasing the exercise capacity of treated patients.

This application claims the benefit under 35 U.S.C. 119(e) of the filing date of Provisional U.S. Application Ser. No. 62/947,416, entitled Devices and Methods for the Creation of an Inter Atrial Shunt for the Treatment of Congestive Heart Failure, filed on Dec. 12, 2019, which application is expressly incorporated herein by reference, in its entirety.

### BACKGROUND OF THE INVENTION

Congestive Heart Failure (CHF) is a major cause of death in the world. In the U.S. alone, about 5.7 million adults suffer from heart failure, and that number is expected to rise as the population continues to age. CHF affects the heart's ability to efficiently pump blood due to stiffening and weakening of the heart muscle. This weakening or stiffening is typically caused by myocardial ischemia, due to a myocardial infarction, cardiomyopathy, or even hypertension. Within a period of five (5) years, 50% of patients diagnosed with heart failure will die. Not only is the disease debilitating for patients, it also costs the U.S. an estimated $30.7 billion per year.

Heart failure is typically split into two different categories, systolic heart failure (SHF) and diastolic heart failure (DHF). In patients suffering from SHF, the left ventricle typically remodels and enlarges to achieve sufficient stroke volume. Although adequate stroke volume may be maintained, the ejection fraction, or the amount of blood pumped out of the ventricle divided by the ventricle volume, decreases to less than 50%. The reduction of ejection fraction leads to an increase in atrial and left ventricle filling pressures.

Systolic function of the left ventricle is typically preserved in patients suffering from DHF. However, the ventricle fails to adequately relax and is unable to fill properly during the diastolic phase. To overcome this, the heart must increase pressure inside the left atrium (LA), leading to increased pulmonary pressures and fluid congestion.

As noted above, one of the debilitating aspects of heart failure is significantly increased LA pressure during exercise. The increase in LA pressures increases the pulmonary pressure, which reduces the amount of blood that can flow through the lungs, resulting in dyspnea or exercise intolerance.

There are several entities pursuing the treatment of heart failure by creating a left-to-right atrium shunt. Corvia and V-Wave are front runners in this space, and have several published studies that demonstrate the safety and efficacy of the treatment. A small study (n=68) that was published in Lancet in 2016 used the Corvia device, showing that 58% of patients treated with a shunt had lower pulmonary pressures at six months and an increase in mean exercise duration. Another more recent safety study of the Corvia device, published in 2018, which was a double-blind, 1:1 sham controlled multicenter trial, demonstrated that the intra atrial shunt was safe, and did not increase any major adverse cardiac, cerebrovascular, or renal events.

Another study utilizing the V-wave interatrial shunt device reported favorable three month results. Ten patients were treated with no adverse events, and had significant improvements in exercise and quality of life assessments. Catheter measurements showed a reduction in the left atrial pressure while maintaining the right heart function. V-wave recently started enrolling in a pivotal study in the U.S. and has received two FDA breakthrough device designations.

### SUMMARY OF THE INVENTION

The present invention is a device and method that can be used to reduce the left atrium pressure by creating an inter-atrial shunt (IAS) between the left and right atrium, thereby reducing pulmonary congestion and resulting in a reduction in pulmonary congestion, thereby reducing symptomatic dyspnea and increasing the exercise capacity of treated patients.

More particularly, in one exemplary aspect of the invention there is disclosed a method of creating an intra-atrial shunt for treatment of congestive heart failure, which comprises steps of inserting a distal tip of a puncture device into a right atrium of a heart and advancing the distal tip of the puncture device through an atrial septum of the heart to form an opening in the atrial septum, and inserting a distal end portion of a guidewire into the right atrium and through the opening in the atrial septum. An additional step includes advancing a portion of a catheter over the guidewire to position a distal portion of the catheter in the right atrium, the distal portion of the catheter having a tapered distal tip and a proximal base, the tapered distal tip of the catheter being moveable between a first position in which the tapered distal tip is disposed at a first distance from the proximal base of the catheter, and a second position in which the tapered distal tip is disposed at a second distance from the proximal base of the catheter, the first distance being greater than the second distance, the catheter having a heater disposed on at least one of the tapered distal tip or the proximal end portion of the catheter. The tapered distal tip of the catheter is advanced through the opening in the atrial septum to dilate the opening and position the tapered distal tip within the left atrium, and the tapered distal tip is moved from the first position to the second position such that a proximal end surface of the tapered distal tip contacts a tissue surface of the atrial septum on the left atrium side of the atrial septum, and a distal end surface of the proximal base of the catheter contacts a tissue surface of the atrial septum on the right atrium side of the atrial septum capturing a tissue portion between the distal end surface of the proximal base of the catheter and the proximal end surface of the distal tip of the catheter. The captured tissue portion is heated with the heater such that a portion of tissue is ablated and an aperture is formed in the atrial septum.

The exemplary method is further characterized with steps of, after the heating, moving the distal tip to the first position within the left atrium, removing the catheter from the heart, and removing the guidewire from the heart. The intra-atrial shunt is devoid of an implant. The proximal surface of the distal tip of the catheter is angled such that when the captured tissue is heated by the heater the aperture formed in the atrial septum is larger than a diameter of the catheter. The proximal surface of the distal tip of the catheter has an angle between 15 and 50 degrees relative to a longitudinal axis of the catheter. The proximal surface of the distal tip of the catheter has an angle relative to a longitudinal axis of the catheter such that a size of the aperture formed in the atrial septum is between about 4-10 mm.

In exemplary methods, the catheter has a heater disposed on both the tapered distal tip and the proximal base of the catheter, and the heating the captured tissue includes heating the captured tissue on each side of the atrial septum. The aperture is preferably formed having smooth edges providing reduced shear stress as fluid flows between the left atrium and the right atrium.

Energy may be supplied to the heater at a temperature between 150 and 600 degrees Celsius, or, more particularly, at a temperature between 150 and 350 degrees Celsius.

In another exemplary aspect of the invention, there is provided an apparatus comprising a catheter having a distal portion configured to be inserted into a heart, the distal portion of the catheter having a tapered distal tip and a proximal base, the tapered distal tip of the catheter being movable between a first position in which the tapered distal tip is disposed at a first distance from the proximal base of the catheter, and a second position in which the tapered distal end portion is disposed at a second distance from the proximal base of the catheter, the first distance being greater than the second distance. The catheter has a heater disposed on at least one of the tapered distal tip or the proximal base of the catheter, and the distal portion of the catheter is configured to capture a portion of tissue of the atrial septum between the distal tip and the proximal base when the distal tip is in the second position. The heater is configured to ablate the captured portion of tissue to form a shunt between the left atrium and the right atrium.

The distal tip of the catheter has a proximal surface, and the proximal base of the catheter has a distal surface, the proximal surface of the distal tip and the distal surface of the proximal base being configured to contact the captured tissue of the atrial septum. The proximal surface of the distal tip of the catheter has an angle between 15 and 50 degrees relative to a longitudinal axis of the catheter. The proximal surface of the distal tip of the catheter has an angle relative to a longitudinal axis of the catheter such that the size of the shunt formed in the captured tissue is between about 4-10 mm.

The catheter has a first heater disposed on the tapered distal tip of the catheter and a second heater disposed on the proximal end portion of the catheter, wherein the first heater and the second heater are configured to ablate the captured tissue on each side of the atrial septum. The distal tip of the catheter includes an outer rib disposed on the proximal end surface, with the outer rib being configured to focus pressure on the captured tissue of the atrial septum to provide for faster cutting and minimal thermal spread. The outer rib of the distal tip of the catheter defines a pocket on the proximal end surface of the distal tip configured to capture tissue ablated by the heater.

The invention, together with additional features and advantages thereof, may best be understood by referencing the following description in conjunction with the accompanying specifications.

### BRIEF DESCRIPTION OF THE DRAWINGS

FIG. 1 is a schematic representation of a general cross-section of the heart, with a guidewire extending through the atrial septum;

FIG. 2a is a view similar to FIG. 1, illustrating a step in one embodiment of the inventive method wherein a catheter having a separable distal tip is tracked over the guidewire in the LA of the heart;

FIG. 2b is a view similar to FIG. 2a, wherein the separable distal tip has been withdrawn proximally to close the tip of the catheter;

FIG. 3 is a view similar to FIG. 2b, illustrating a cross-section of the heart, with a hole in the atrial septum created by the catheter;

FIG. 4a is a schematic view of an exemplary embodiment of a catheter constructed in accordance with the principles of the present invention;

FIG. 4b is an enlarged view of an exemplary embodiment of the separable distal tip of the catheter shown in FIG. 4a, in a closed orientation;

FIG. 5 is an enlarged view of the separable distal tip of the catheter shown in FIG. 4b, in an open orientation;

FIG. 6 is an isometric view of the distal tip of the catheter; and

FIG. 7 is a view illustrating a particular heating device usable with the present invention.

### DETAILED DESCRIPTION OF THE INVENTION

### Notation and Nomenclature

Certain terms are used throughout the following description and claims to refer to particular system components. As one skilled in the art will appreciate, companies that design and manufacture medical devices may refer to a component by different names. This document does not intend to distinguish between components that differ in name but not function.

In the following discussion and in the claims, the terms “including” and “comprising” are used in an open-ended fashion, and thus should be interpreted to mean “including, but not limited to . . . .”

Reference to a singular item includes the possibility that there are plural of the same items present. More specifically, as used herein and in the appended claims, the singular forms “a,” “an,” “said” and “the” include plural references unless the context clearly dictates otherwise. It is further noted that the claims may be drafted to exclude any optional element. As such, this statement serves as antecedent basis for use of such exclusive terminology as “solely,” “only” and the like in connection with the recitation of claim elements, or use of a “negative” limitation. Lastly, it is to be appreciated that unless defined otherwise, all technical and scientific terms used herein have the same meaning as commonly understood by one of ordinary skill in the art to which this invention belongs.

Where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent application and hardware) is expressly incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Before the various embodiments are described in detail, it is to be understood that this invention is not limited to particular variations set forth herein as various changes or modifications may be made, and equivalents may be substituted, without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

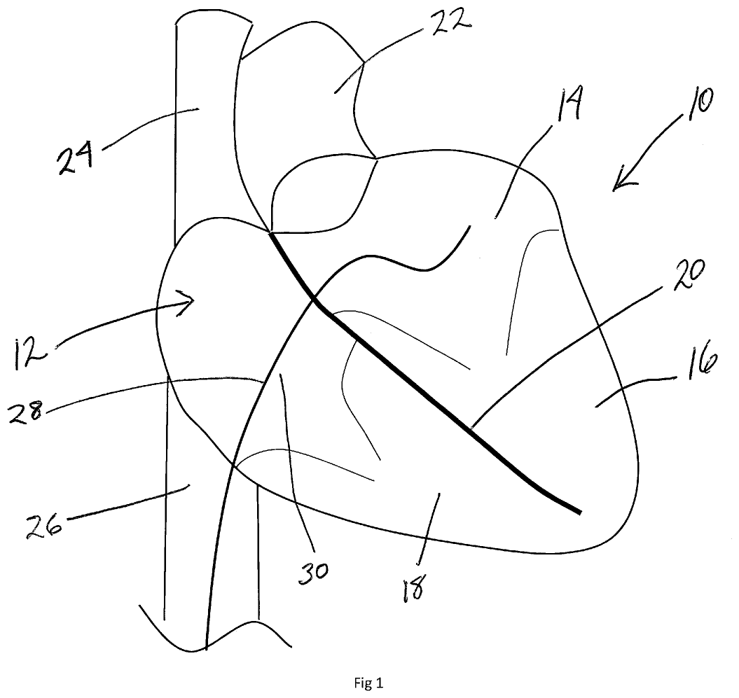

Referring now more particularly to the drawings, FIG. 1 illustrates the anatomy of a heart 10 of a patient, which includes a right atrium 12, a left atrium, 14, a left ventricle 16, and a right ventricle 18, with the left and right sides of the heart 10 being divided by a septum 20. Also illustrated are the aorta 22, the superior vena cava 24, and the inferior vena cava 26. In FIG. 1, as a first step of an exemplary method according to the invention, a guidewire 28 has been placed within the heart, as illustrated, through the right atrium 12 and the Fossa Ovalus 30, through the atrial septum 20, and into the left atrium 14. The septum 20 may be punctured using standard techniques, such as a Brochenbraugh needle.

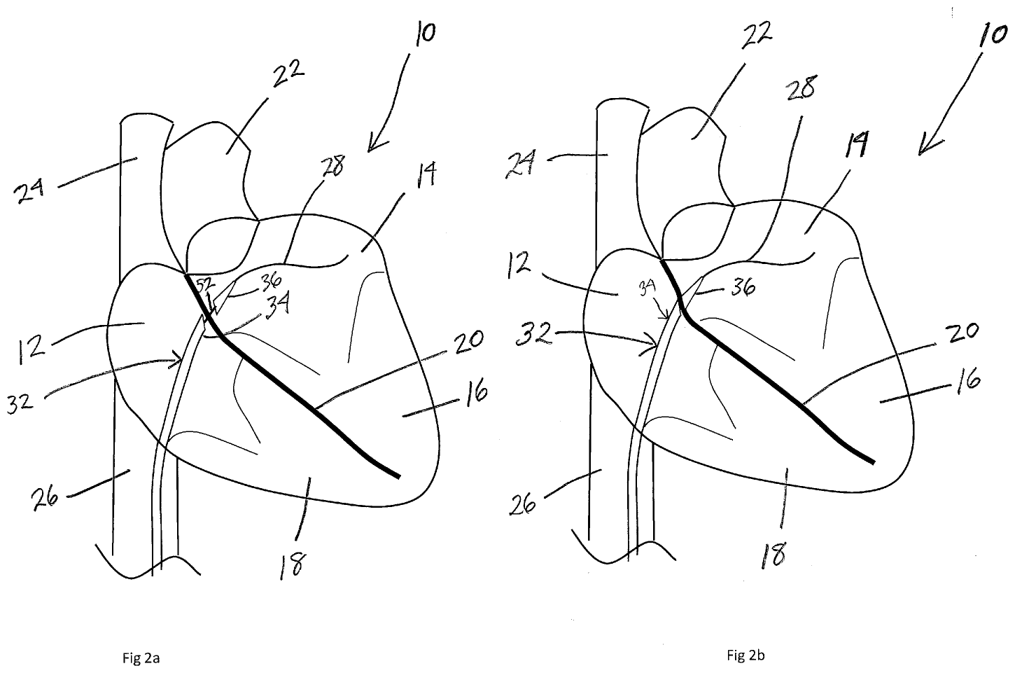

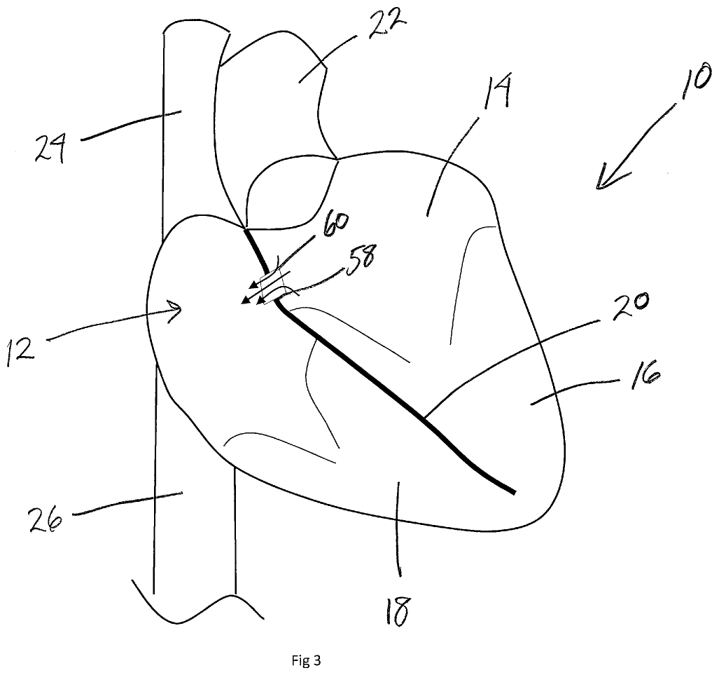

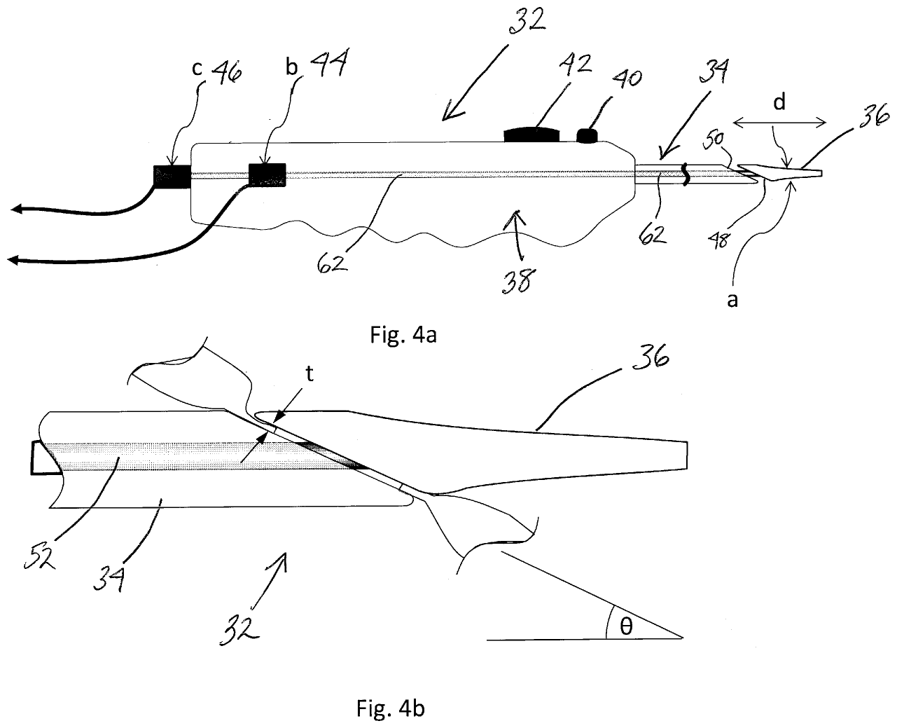

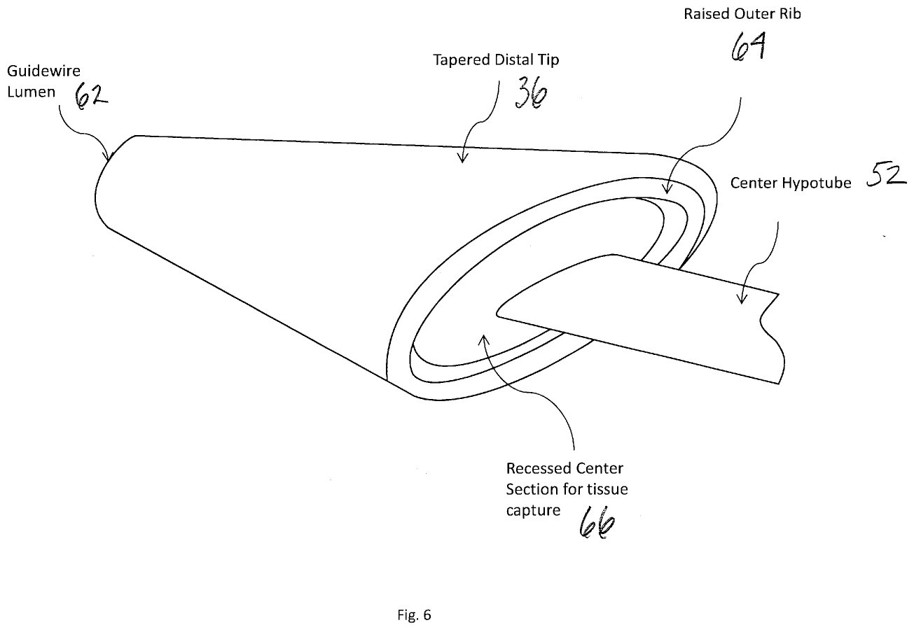

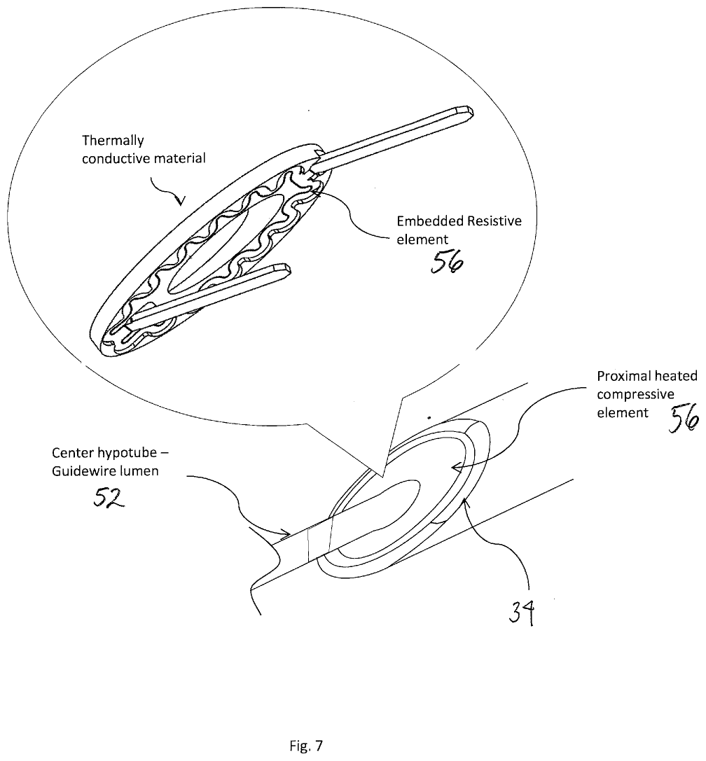

Now, as shown in FIGS. 2a, 2b, and 3, a catheter 32 is tracked over the guidewire 28 to the desired procedural site. The catheter may be of the type disclosed in commonly assigned U.S. Pat. Nos. 9,439,710, 9,452,015, 9,474,562, and 10,772,672, herein expressly incorporated by reference, in their entirety, and currently sold under the trademark ELLIPSYS® by the assignee, Avenu Medical, Inc. of San Juan Capistrano, Calif. The catheter 32 is shown in more detail in FIGS. 4a, 4b, and 5-7, and comprises a proximal portion 34 and a distal tip 36. A handle 38 is disposed at the proximal end of the proximal portion 34 (FIG. 4a). In an exemplary embodiment, the handle 38 may comprise a button actuator 40, a slide actuator 42, a main body lumen Luer 44, and an electrode lumen Luer 46.

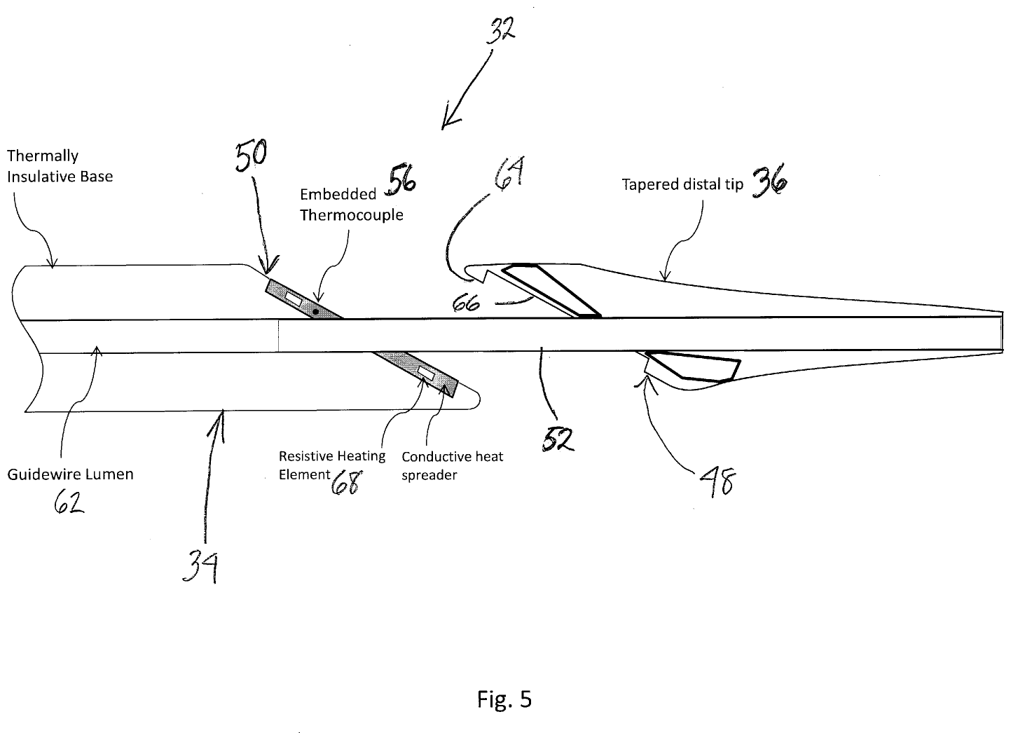

As noted above, the catheter 32 comprises a proximal portion 34, which may be constructed to be flexible distally of the handle 38, and a distal tip 36. The distal tip 36 is movable between a closed position, wherein a proximal surface 48 of the distal tip 36 is in contact with a distal surface 50 of the proximal portion 34, as illustrated in FIG. 4b, and an open position, wherein the distal tip 36 is distally spaced from the proximal portion 34, as shown in FIG. 5. The slide actuator 42 may be utilized to actuate the distal tip 36 to move between the open and closed positions. A shaft 52 secures the distal tip 36 to the proximal portion 34, and permits the aforementioned relative sliding movement.

Referring again to FIGS. 2a, 2b, and 3, at the procedural site, the tapered distal tip 36 is advanced relative to the proximal portion 34, using, for example, the actuator 42 (FIG. 4a), and dilates through the septum wall 20, wherein a small aperture had previously been created to accommodate the guidewire 28. The distal tip 36 is advanced forwardly into the left atrium 14 until the angled face 50 of the proximal portion 34 engages the septum wall 20. The angle θ between the distal tip 36 and the proximal portion 34 creates an aperture larger than the diameter of the catheter 32. This angle is preferably about 15-50 degrees relative to the longitudinal axis. In one particularly optimal configuration, the angle is approximately 23 degrees relative to the longitudinal axis.

A barb at the distal end of the proximal portion 34 may be provided to prevent the proximal portion 34 from advancing through the septum wall 20. The catheter may also be pre-shaped (curved), as shown in the figures, to assist advancement of the catheter.

While the catheter is shown in its open configuration in FIG. 2a, wherein the distal tip 36 is spaced from the proximal portion 34, in FIG. 2b the distal tip 36 has been withdrawn proximally to its closed configuration, wherein the distal tip proximal surface 48 and proximal portion distal surface 50 provide pressure on the septum wall tissue captured between the elements. A heater 56, such as the illustrated embedded thermocouple shown in FIG. 5, may be provided on either the proximal portion distal surface 50, as shown, or the distal portion proximal surface 48, or both, in order to provide a heated surface controlled to ablate tissues captured between the two angled surfaces.

Once the captured tissue 20 has been fully ablated, forming an opening 58 through the septum wall 20, as shown in FIG. 3, the catheter 32 and guidewire 28 are removed from the heart 10. Consequently, blood flows through the opening 58, from the left atrium into the right atrium, as illustrated by arrows 60, thereby reducing pressure in the left atrium.

As noted above, FIGS. 4a, 4b, and 5-7 illustrate various features of the catheter 32 which might be used in connection with the inventive methods discussed herein. In particular, the catheter 32 is equipped with a catheter lumen 62, which may be accommodated within the shaft or hypotube 52. A preferred catheter size is 6-12F, with a flexible shaft, as noted above. The distal tip 36 is preferably tapered, hydrophilic coated, and equipped with radiopaque markers for imaging purposes. The angle θ between the tip and base, discussed above, is optimized to match entry from the IVC, and can be varied to change the length of the shunt. In illustrated embodiments, this length may be adjusted to between about 4-10 mm. A sensor is provided to monitor the amount of tissue captured between the elements, measuring distance between the respective surfaces 48 and 50, to verify correct placement and amount of energy to deliver in order to ablate the captured tissue.

A raised outer rib 64 on the distal tip 36 focuses pressure on the heated surface 56 of the proximal portion 34. This allows faster cutting and minimal thermal spread. As noted above, the tip is tapered so that it can dilate through the septum 20. The hypotube/shaft 52 may be offset from the center of the catheter tube to bias the rib from one side to the other (not shown), if desired. The offset may help to engage tissue if the device is used for multiple passes to enlarge the opening 58.

The rib 64 defines a recessed tissue capture pocket 66 for capturing ablated tissue. Pressure is focused on the heater to quickly cut the septum 20.

A resistive heating element 68 is embedded in a heat spreader 70 formed of a conductive material, to provide uniform heating of the tissue, with minimal thermal spread and a minimal heating surface. Thermally conductive materials may be anodized aluminum, ceramic, or other steel. The thermocouple 56 may also be embedded in the conductive material 70 to provide closed loop thermal control. Heating regimes may be monopolar or bipolar, depending upon application.

In certain circumstances, heating pulses may be preferred, wherein duration is modified to control thermal spread into adjacent tissue. A control consideration is that, in some instances, more thermal spread may increase scarring and reduce chance of closure. It is preferred to heat from the right atrium, which is the low pressure side, to minimize the chance of arterial emboli, which would be pushed from the LA into the RA.

Pressure in the lung and LA can be measured post-shunt creation. If the pressure is still higher than desired, then the size of the shunt can be increased by reinserting device 32 and ablating another section of tissue or increasing the size of the shunt with a balloon catheter.

An advantage of the present approach is that no implant is required or left behind, thereby reducing the risk of thrombus formation. The opening 58 can therefore be closed or reversed easier with standard off-the-shelf atrial septum closure devices.

Accordingly, although an exemplary embodiment and method according to the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A method of creating an intra-atrial shunt for treatment of congestive heart failure, comprising:
inserting a distal tip of a puncture device into a right atrium of a heart and advancing the distal tip of the puncture device through an atrial septum of the heart to form an opening in the atrial septum;
inserting a distal end portion of a guidewire into the right atrium and through the opening in the atrial septum;
advancing a portion of a catheter over the guidewire to position a distal portion of the catheter in the right atrium, the distal portion of the catheter having a tapered distal tip and a proximal base, the tapered distal tip of the catheter being moveable between a first position in which the tapered distal tip is disposed at a first distance from the proximal base of the catheter, and a second position in which the tapered distal tip is disposed at a second distance from the proximal base of the catheter, the first distance being greater than the second distance, the catheter having a heater disposed on at least one of the tapered distal tip or the proximal end portion of the catheter;
advancing the tapered distal tip of the catheter through the opening in the atrial septum to dilate the opening and position the tapered distal tip within the left atrium;
moving the tapered distal tip from the first position to the second position such that a proximal end surface of the tapered distal tip contacts a tissue surface of the atrial septum on the left atrium side of the atrial septum and a distal end surface of the proximal base of the catheter contacts a tissue surface of the atrial septum on the right atrium side of the atrial septum capturing a tissue portion between the distal end surface of the proximal base of the catheter and the proximal end surface of the distal tip of the catheter; and
heating the captured tissue portion with the heater such that a portion of tissue is ablated and an aperture is formed in the atrial septum.

2. The method of claim 1, further comprising:
after the heating, moving the distal tip to the first position within the left atrium;
removing the catheter from the heart; and
removing the guidewire from the heart.

3. The method of claim 1, wherein the intra-atrial shunt is devoid of an implant.

4. The method of claim 1, wherein the proximal surface of the distal tip of the catheter is angled such that when the captured tissue is heated by the heater the aperture formed in the atrial septum is larger than a diameter of the catheter.

5. The method of claim 4, wherein the proximal surface of the distal tip of the catheter has an angle between 15 and 50 degrees relative to a longitudinal axis of the catheter.

6. The method of claim 1, wherein the proximal surface of the distal tip of the catheter has an angle relative to a longitudinal axis of the catheter such that a size of the aperture formed in the atrial septum is between about 4-10 mm.

7. The method of claim 1, wherein the catheter has a heater disposed on both the tapered distal tip and the proximal base of the catheter, and
the heating the captured tissue includes heating the captured tissue on each side of the atrial septum.

8. The method of claim 1, wherein the aperture is formed having smooth edges providing reduced shear stress as fluid flows between the left atrium and the right atrium.

9. The method of claim 1, further comprising:
supplying energy to the heater at a temperature between 150 and 600 degrees Celsius.

10. The method of claim 9 wherein the supplying energy step comprises supplying energy to the heater at a temperature between 150 and 350 degrees Celsius.

11. An apparatus, comprising:
a catheter having a distal portion configured to be inserted into a heart, the distal portion of the catheter having a tapered distal tip and a proximal base, the tapered distal tip of the catheter being movable between a first position in which the tapered distal tip is disposed at a first distance from the proximal base of the catheter, and a second position in which the tapered distal end portion is disposed at a second distance from the proximal base of the catheter, the first distance being greater than the second distance,
the catheter having a heater disposed on at least one of the tapered distal tip or the proximal base of the catheter;
the distal portion of the catheter configured to capture a portion of tissue of the atrial septum between the distal tip and the proximal base when the distal tip is in the second position; and
the heater configured to ablate the captured portion of tissue to form a shunt between the left atrium and the right atrium.

12. The apparatus of claim 11, wherein the distal tip of the catheter has a proximal surface and the proximal base of the catheter has a distal surface, the proximal surface of the distal tip and the distal surface of the proximal base configured to contact the captured tissue of the atrial septum.

13. The apparatus of claim 12, wherein the proximal surface of the distal tip of the catheter has an angle between 15 and 50 degrees relative to a longitudinal axis of the catheter.

14. The apparatus of claim 12, wherein the proximal surface of the distal tip of the catheter has an angle relative to a longitudinal axis of the catheter such that the size of the shunt formed in the captured tissue is between about 4-10 mm.

15. The apparatus of claim 11, wherein the catheter has a first heater disposed on the tapered distal tip of the catheter and a second heater dispose on the proximal end portion of the catheter, the first heater and the second heater being configured to ablate the captured tissue on each side of the atrial septum.

16. The apparatus of claim 11, wherein the distal tip of the catheter includes an outer rib disposed on the proximal end surface, the outer rib is configured to focus pressure on the captured tissue of the atrial septum to provide for faster cutting and minimal thermal spread.

17. The apparatus of claim 11, wherein the outer rib of the distal tip of the catheter defines a pocket on the proximal end surface of the distal tip configured to capture tissue ablated by the heater.
